(19) United States
(12) Patent Application Publication
(10) Pub. No.: US 2004/0243224 A1
(43) Pub. Date: Dec. 2, 2004

(54) Methods and compositions for inhibiting narrowing in mammalian vascular pathways

(75) Inventor: Patrice Tremble, Santa Rosa, CA (US)

Correspondence Address: STRADLING YOCCO CARLSON & RAUTH, SUITE 1600, 660 NEWPORT CENTER DRIVE, P.O. BOX 7680, NEWPORT BEACH, CA, 92660, US

(73) Assignee: Medtronic Vascular, Inc., Santa Rosa, CA

(21) Appl. No.: 10/818,786

(22) Filed: Apr. 5, 2004

Related U.S. Application Data
(60) Provisional application No. 60/460,366, filed on Apr. 3, 2003.

(51) Int. Cl. A61F 2/06

(52) U.S. Cl. 623/1.42; 514/44

(57) ABSTRACT

Methods and related compositions for treating vascular occlusions and preventing vascular narrowing are disclosed. In one embodiment an implantable medical device is provided with a coating comprising at least one cell growth inhibiting ubiquitin activator. In another embodiment a micro syringe is provided that injects the cell growth inhibiting ubiquitin activator directly into the adventitia. One specific embodiment includes a vascular stent having a cell growth inhibiting ubiquitin activator, specifically, hypothemycin.

### RELATED APPLICATIONS

The present application claims priority to U.S. provisional patent application Serial No. 60/460,366 filed Apr. 5, 2003, the entire contents of which is incorporated by reference.

### FIELD OF THE INVENTION

The present invention provides methods and compositions for the inhibition of vascular narrowing in mammalian vascular pathways. More particularly, the present invention provides methods and associated compositions for inhibiting vascular narrowing by inhibiting cell growth in vascular tissues at specific sites.

### BACKGROUND OF THE INVENTION

The mammalian vascular system provides pathways for the critical flow and transport of water, nutrient fluids, and oxygenated blood to the heart, tissues and other organs in the body. These vascular pathways, commonly referred to as arteries and veins, can become narrowed over time due to disease or injury. For example, cardiovascular disease, is a serious health problem caused by narrowing of the coronary arteries that provide blood to the heart. Vascular narrowing of the coronary arteries is one of the major contributors to human illness in developing and industrialized countries. In circumstances where cardiovascular disease results in the total blockage or “occlusion” of a coronary artery, cell death in the heart muscle can result due to the lack of oxygenated blood being delivered downstream of the occlusion. This heart muscle cell death is what is commonly referred to as a heart attack and can have serious life limiting consequences including death. Cigarette smoking, high blood-cholesterol levels, hypertension, diabetes, physical inactivity and obesity have been identified as major risk factors associated with and even contributing to cardiovascular disease and vascular narrowing.

The contemporary medical treatment of vascular narrowing begins with its identification and diagnosis by experienced medical professionals. Vascular narrowing is commonly identified with known diagnostic procedures such as X-rays, ultrasound imaging, direct inspection and observation with catheter endoscopic devices, fluoroscope observation of injected dyes, echocardiograms, and other imaging techniques.

For example, using x-rays to observe a dye highlighting the interior of a vascular pathway enables a cardiologist to identify specific areas evidencing vascular narrowing in a relatively simple, outpatient procedure known as an “angiogram”. First, the cardiologist inserts a thin tube, or catheter, into an artery leading to the heart by making an incision into the appropriate artery in the groin or in the arm of the patient and then advancing the catheter within the artery to a position close to the patient's heart. Next, the cardiologist injects a dye through the positioned catheter into the artery so the interior of the coronary arteries show up on x-rays. By taking a series of X-rays while the dye is in the arteries the cardiologist is able to directly visualize any vascular narrowing and to identify the site or sites of such narrowing in the coronary arteries.

After diagnosis has been made and the target site or sites have been identified, there are several treatment options available to the cardiologist for treating narrowed arteries to restore blood flow before a heart attack occurs. Vascular surgery through conventional open heart techniques or through less invasive angioplasty procedures remain the primary treatments available for reopening vascular narrowing in coronary arteries. Significant lifestyle changes may be prescribed as well, modifying the patient's diet and exercise patterns to assist in reducing the chances of further vascular narrowing.

Conventional open-heart surgery requires opening of the patient's chest to access coronary arteries in order to bypass narrowed or blocked sites in the coronary arteries. Coronary bypass is accomplished by harvesting a section of healthy vein, typically from the patient's thigh. One end of this section of harvested vein is then grafted into the blocked artery upstream of the vascular narrowing or “stenosis” to form the beginning of an alternate blood pathway around the stenotic blockage or lesion. The other end of the harvested vein is grafted to the blocked artery downstream of the blockage, creating an open blood pathway bypassing the blocked or narrowed stenotic lesion and restoring essential blood flow around the lesion. Though a common medical procedure, coronary artery bypass grafting is not without serious risks and potential complications. At a minimum, an extended stay in the hospital is required for the patient to recover from surgery.

A more recently developed, less invasive alternative medical treatment option for restoring blood flow to blocked or narrowed coronary arteries is called “angioplasty” or “balloon angioplasty”. In essence, balloon angioplasty involves directly opening the stenotic lesion of the blocked or narrowed artery from the inside of the artery and does not involve opening the patient's chest. Typical angioplasty procedures utilize coronary catheters to access coronary arteries much like those involved with accessing the heart during an angiogram. For example, first, as with an angiogram procedure, the cardiologist uses X-ray visualization to directly observe the coronary arteries as a narrow catheter having an expandable balloon at its end, commonly called a “balloon catheter” is threaded into the blocked vessel so that the deflated balloon on the end of the catheter is positioned directly across the narrowed stenotic lesion. Then the cardiologist inflates the balloon with a liquid such as sterile saline solution to expand the balloon within the narrowed lesion. Expanding the balloon compresses the “plaque build-up”, unwanted deposits or cell growth forming the narrowing within the artery, outwardly against the walls of the artery. Displacing the plaque buildup toward the walls of the artery restores the “patency” or openness of the blocked vessel so that blood will again flow through the vessel, maintaining the health of the tissues downstream of the stenosis.

Balloon angioplasty has become increasingly common as it is minimally invasive and does not carry the risks associated with open-heart surgery. Further, angioplasty patients rarely require extended hospitalization. Not only is this a benefit to the patient, but it also significantly reduces the expenses associated with opening blocked coronary arteries. Though remarkably successful, angioplasty is not without its own risks and complications. One of these complications is known as “restenosis” where the opened artery renarrows following the procedure. This renarrowing can occur suddenly after the angioplasty procedure as in situations known as “acute restenosis” or it can occur over a period of months following the initial balloon angioplasty.

In response to this problem of restenosis an additional medical technique utilizing a “stent” may be employed to keep the artery open. A stent is a mechanical structure typically formed of an expandable metal mesh tube or sleeve. A stent is positioned in a narrowed, small diameter form across the stenotic lesion that was the target site of the angioplasty. Once positioned across the target site the stent is expanded by inflating a balloon catheter threaded through the stent to increase the diameter of the stent in order to mechanically hold the vessel open at the site of the blockage or narrowing. Stents are being used increasingly in combination with balloon catheters as part of the initial opening procedure after a heart attack in conjunction with balloon angioplasty in order to resist restenosis.

Stenting is now used in many angioplasty procedures. Recent studies report higher survival rates associated with the use of stents, including their use within multiple blood vessels having multiple target sites or stenotic lesions. Most, but not all, patients are suitable candidates for stents. However, a more recent complication has been identified in connection with stent usage. In some cases restenosis occurs even after a stent has been positioned across a narrowed or blocked stenotic lesion. Restoring vessel patency after a stent has been positioned can be a difficult procedure because balloon angioplasty may no longer be possible due to the expanded stent positioned across the lesion possibly interfering with the ability of the balloon to expand and compress the stenotic lesion.

The success of any vascular re-opening procedure, referred to as “revascularization”, depends largely on the initial openness or patency of the vascular site subject to the vessel opening procedure being performed. Still, for approximately 30-60% of the patients treated with balloon angioplasty and stent deployment, injury to the vessel walls may result from the expansion of the balloon which can cause microscopic tearing to the tissues forming the layers of the vascular wall. This tearing can lead to the subsequent growth of scar tissue which itself may cause renarrowing of the vessel.

Such post-angioplasty restenosis is one of the major factors limiting the long-term success of angioplasty procedures utilized to restore the patency of coronary arteries and other vascular pathways. The risk of vascular narrowing or restenosis caused by unwanted cell growth is a complication that has not been fully addressed by the medical techniques of the prior art. Recent attempts at dealing with restenosis have included the use of known anti-restenotic drugs, oral blood modification therapies to thin the blood and increase blood flow through narrowing lesions, and the application of heat or radioactive agents to stenotic vascular sites to kill cells and prevent their growth. Though generally successful, each of these techniques has its own drawbacks.

The major drawback associated with anti-restenotic drug therapy is developing or selecting the appropriate drug or drug mixture that will act positively on the interior of the vascular pathway without negative side effects. Further, delivering effective amounts of such drugs to the target sites is difficult. Additionally, known anti-restenotic drugs only act on inner walls of the vascular pathway and do not medically treat injuries to other layers of the vascular wall that may be involved in restenosis.

Limitations associated with known oral blood modification or blood-thinning therapies include the potential for blood leakage and uncontrollable bleeding in response to subsequent injuries or traumatic accidents much like those associated with the natural genetic condition know as hemophilia. Further, potentially serious circumstances can occur where such systemic agents can cause adverse effects to other organs of the patient's body.

Similarly, the use of radioactive isotopes placed in the human blood circulatory system or in sensitive tissues adjacent to vascular pathways can be complicated and costly. Radioactive isotopes must be handled with care by medical personnel and have been known to cause trauma to healthy tissues adjacent to targeted sites in narrowed vascular pathways. These and other drawbacks can discourage the use of such anti-restenosis treatments.

The present invention overcomes these obstacles and drawbacks by providing novel methods and compositions that inhibit vascular narrowing and renarrowing.

### SUMMARY OF THE INVENTION

These and other objects are achieved by the present invention which provides, in a broad aspect, methods and medicament compositions for inhibiting vascular narrowing in mammalian patients by inhibiting cell growth at the target site or sites exhibiting vascular narrowing or the potential for vascular narrowing or renarrowing. In accordance with the teachings of the present invention, restraining or inhibiting unwanted cell growth is accomplished by delivering at least one cell growth inhibiting ubiquitin activator to an identified target vascular site. More specifically, the present invention provides methods for inhibiting vascular narrowing in mammals by first identifying a target vascular site or sites at risk of narrowing, and then delivering at least one cell growth inhibiting ubiquitin activator to the identified target vascular sites.

The cell growth inhibiting ubiquitin activators of the present invention are selected from the group consisting of protein kinase inhibitors, tyrosine-specific kinase inhibitors, deubiquitination enzyme inhibitors, antibiotics, antifungal agents, anti-tumor agents and derivative products thereof. For example, an exemplary cell growth inhibiting ubiquitin activator of the present invention is hypothemycin. Hypothemycin is commonly known in the art as an antibiotic. In accordance with the teachings of the present invention, hypothemycin is utilized as a cell growth inhibiting ubiquitin activator that is delivered to one or more target sites that have been identified as exhibiting narrowing, or as being at risk of narrowing. Once delivered to target sites, hypothemycin inhibits cell growth by reacting with at least one biologic factor involved with cell growth regulation or promotion. One of these biologic factors is called “ubiquitin” which is a protein found in virtually all-mammalian cells. Hypothemycin reacts with ubiquitin in a variety of ways to restrain or inhibit cell growth including activating ubiquitin to promote cell growth degrading processes which in turn discourage cell growth. When proteins degrade over time, this is called “protein-turnover”. In this manner, hypothemycin inhibits vascular narrowing by inhibiting cell growth at the target site to which it has been delivered in accordance with the teachings of the present invention.

An additional aspect of the methods and medicament compositions of the present invention includes delivering at least one cell growth inhibiting ubiquitin activator to specific tissue or tissues of identified target vascular sites. In accordance with the teachings of the present invention, delivering compositions of the present invention to the target vascular site or sites can be achieved by positioning delivering apparatus at the target sites, either within the vascular pathway itself or directly adjacent thereto, for delivering at least one cell growth inhibiting ubiquitin activator. Exemplary delivering apparatus within the scope and teachings of the present invention include catheters, stents, microsyringes and other endoscopic transport devices. It is also within the scope of the present invention to utilize delivering apparatus such as syringes, IV's, and drug eluting implants for delivering the compositions of the present invention to the identified target sites through the patient's skin by directly positioning such apparatus adjacent to the target sites. The present invention also utilizes simple and minimally invasive endoscopic or catheter-based microsyringe techniques for delivering compositions from within the vascular pathways themselves.

These exemplary activator delivering apparatus are able to deliver at least one cell growth inhibiting ubiquitin activator to target sites from within the narrowed vascular pathways because they are positioned adjacent to the lesion at the target sire or sites before the compositions of the present invention are released. For example, in accordance with the teachings of the present invention, a catheter having an infusion tip at or near its distal end is advanced to the target site such that the infusion tip is positioned just at or upstream of the target site. Then compositions of the present invention that have been compounded with suitable carriers are pumped through the catheter for release at the infusion tip and delivering to the target site or sites where the compositions are absorbed by the surrounding tissues of the target vascular pathway.

Alternatively, it is also within the scope of the present invention for the infusion tip of the catheter to incorporate a porous balloon that is inflated with the appropriately compounded cell growth inhibiting ubiquitin activator compositions of the present invention after the porous balloon of the infusion tip has been positioned at or near the target site or sites. In this manner, one or more compositions of the present invention escapes through the pores of the inflated balloon and are delivered to the internal tissues of the vascular pathway at the target sites.

It is also within the scope of the present invention to utilize a catheter to position a controlled release drug-eluting stent across the lesion to deliver at least one cell growth inhibiting ubiquitin activator to the internal surfaces of the patient's vascular pathway at the target site or sites. Utilizing this delivering technique of the present invention requires that the exemplary compositions of the present invention be compounded for release from a drug eluting coating, attachment or film physically associated with the delivering stent apparatus. Accordingly, the medicament compositions of the present invention can be compounded in appropriate dosages with dissolving polymers, terpolymers, hydrogels, salts, or other controlled release drug eluting compounds that will form stable coatings on one or more surfaces of the exemplary drug releasing stents such that the drugs will be released at the target site following placement of the stent at the target site.

Alternately, compounded medicament compositions of the present invention are incorporated into grooves or wells provided in the delivering apparatus while the stent is in its collapsed state. The delivering of the medicament compositions of the present invention to identified targets sites is accomplished when the stent is positioned at or near the site and then expanded, opening the grooves or wells to release the compounded medicament compositions of the present invention at the target site.

An additional exemplary embodiment of the present invention utilizes a microsyringe for delivering compounded medicament compositions of the present invention into specific tissues at identified target site or sites within the patient's vasculature. By compounding the medicament compositions of the present invention for injection with an appropriate liquid or viscous carriers it is possible to inject the medicament compositions of the present invention directly into specific vascular tissues of the vascular pathway in order to inhibit cell growth within these specific tissues at the identified target sites.

As those skilled in the art will appreciate, a vascular microsyringe is a balloon catheter based delivering apparatus provided with a tiny hollow needle at its distal end and is an example of medical delivering devices within the scope of the present invention. Following its positioning at identified target sites within the patient's vasculature, the catheter is pressurized by inflating it with a compounded composition or compositions of the present invention. Inflating the positioned catheter forces the microsyringe into position where its sharpened tip is exposed and penetrates the internal surface of the patient's blood vessel and then delivers the compounded medicament composition to the tissue or tissues forming the layers of the vascular wall. Configuring the needle of the microsyringe with the appropriate dimensions makes possible the delivery of an effective amount of the medicament composition or compositions of the present invention directly to specific tissue layers of the patient's vasculature. This aspect of the present invention greatly reduces the amount of drug needed to accomplish the inhibition of vascular narrowing and reduces the side effects associated with unnecessarily delivering the medicaments of the present invention to other tissues in the patient's body.

The further illustrates an additional advantage of the present invention over the prior art, the ability of the present invention to deliver at least one cell growth inhibiting ubiquitin activator to a specific tissue or tissues at target vascular sites as opposed to the prior art's more generalized systemic delivering techniques to all bodily tissues. As a result, the present invention is able to inhibit vascular narrowing by inhibiting cell growth in specific vascular tissues responsible for cell growth associated with vascular narrowing and renarrowing at specific target sites. This reduces the possibility of unwanted side effects resulting from less specific drug delivery to tissues that may not be involved with vascular narrowing or renarrowing. For example, in accordance with the teachings of the present invention, at least one cell growth inhibiting ubiquitin activator is delivered into the vascular adventitial layer.

The vascular adventitial layer or “adventitia” is one of the tissue layers that make up the walls of a vein or artery. As such it contains a network of blood vessels that transport nutrients, fluids and oxygenated blood to the vascular pathways themselves. Injection of the cell growth inhibiting ubiquitin activators of the present invention into the vascular adventitial layer, in addition to overcoming many of the drawbacks of the prior art, also makes it possible to use much smaller dosages of the cell growth inhibiting ubiquitin activators without sacrificing the effectiveness of the treatment methods. Those skilled in the art also will appreciate that reducing the quantity of pharmaceutical compounds delivered to a patient has added benefits including reduced costs as well as reduced side effects.

Other objects, features, and advantages of the present invention will be apparent to those skilled in the art from a consideration of the following detailed description of exemplary embodiments thereof.

### BRIEF DESCRIPTION OF THE DRAWINGS

FIG. 1 is a partial cut-away view of an exemplary vascular pathway 10 with three vascular tissue layers identified as intimal layer 12, medial layer 14 and adventitial layer 16 and vascular narrowing evidenced by unwanted cell growth and “plaque build-up” 18 on the interior surface of intimal layer 12.

FIG. 2 is a partial cross-sectional view of vascular pathway 10 of FIG. 1 illustrating delivering apparatus microsyringe 28 carried on the surface of actuating balloon 26 with distal end 22 and proximal end 24 positioned by catheter 20 at a vascular target site identified by plaque build-up 18.

FIG. 3 is a partial cross-sectional view of vascular pathway 10 of FIG. 2 illustrating actuating balloon 26 inflated and microsyringe 28 and microsyringe tip 29 penetrating through intimal layer 12, medial layer 14 into adventitial layer 16 delivering at least one medicament composition of the present invention to adventitial layer 16 as drug flow 30.

FIG. 4 is a partial cross-sectional view of vascular pathway 10 of FIG. 1 illustrating actuating balloon 26 with distal end 22 and proximal end 24 positioned by catheter 20 at a vascular target site identified by plaque build-up 18 and delivering apparatus drug eluting stent 32 carried on the surface of actuating balloon 26.

FIG. 5 is a partial cross-sectional view of vascular pathway 10 of FIG. 4 illustrating actuating balloon 26 inflated to expand drug eluting stent 32 in vascular pathway 10 and displace plaque build-up 18 and deliver at least one medicament composition of the present invention as illustrated by drug flow 30.

FIG. 6 is an illustrative example of the chemical structure of hypothemycin and a related derivative product within the scope and teachings of the present invention.

### DETAILED DESCRIPTION OF THE INVENTION

The present invention provides methods and associated medicament compositions for inhibiting vascular narrowing in mammalian patients. Utilizing the teachings of the present invention it is now possible to reduce the incidence of and the risks associated with vascular narrowing and renarrowing, as well as to reduce the complications associated with contemporary treatment methodologies for vascular narrowing such as coronary bypass. Unlike the prior art, the present invention accomplishes these beneficial objectives by inhibiting unwanted cell growth at an identified target site or sites exhibiting vascular narrowing or the potential for vascular narrowing. At such target vascular sites there is a need to specifically inhibit further unwanted cell growth without negatively impacting tissues at other parts of the patient's body. This can be accomplished through the methods and medicament compositions of the present invention by inhibiting or restraining the ability of cell growth factors to induce further unwanted cell growth in specific tissues at these target site or sites.

In accordance with the teachings of the present invention, inhibiting unwanted cell growth is accomplished through relatively simple, minimally invasive procedures that can be repeated if necessary. Specifically, the present invention provides, in a broad aspect, methods for inhibiting vascular narrowing in mammals by first identifying one or more target vascular sites exhibiting narrowing or the risk of narrowing, and then delivering an anti-proliferative amount at least one cell growth inhibiting ubiquitin activator to tissues at the target vascular site or sites. An anti-proliferative amount is defined herein as the amount of a cell growth inhibitor (anti-proliferative) that prevents vascular smooth muscle cell growth to the extent necessary to treat vascular narrowing and/or prevent restenosis. Persons having ordinary skill in the art will be able to ascertain anti-proliferative concentrations of the cell growth inhibiting ubiquitin activators of the present invention without undue experimentation using method known in the art or pharmacology. Generally, and not intended as a limitation the cell growth inhibiting ubiquitin activators of the present invention are administer at a concentration of between 0.1 μg per mL to 100 mg per mL if administered in liquid form (via a micro injection catheter or balloon catheter). If delivered from an implantable medical device such as a vascular stent, the device coating will comprise between 1 μg to 1 g of cell growth inhibiting ubiquitin activator. The cell growth inhibiting ubiquitin activator medicament compositions of the present invention are selected from the group consisting of protein kinase inhibitors, tyrosine-specific kinase inhibitors, deubiquitination enzyme inhibitors, antibiotics, antifungal agents, and anti-tumor agents; and derivative products thereof.

More particularly, the first step of the present invention is the identification of at least one target site within the patient's vasculature that exhibits vascular narrowing. Such sites can include vessels exhibiting some degree of blockage or occlusion, or sites suspected of having the potential for subsequent narrowing or blockage. These target sites can include vascular pathways with torn or scarred wall tissues that may be prone to narrowing. This first step is accomplished using a variety of diagnostic imaging procedures as known or developed in the art. Typically, trained physicians practice these diagnostic imaging procedures and utilize their experience and judgment to identify and locate vascular sites in need of prophylactic or therapeutic treatment to reduce the chances of serious vascular narrowing in a patient's vasculature .

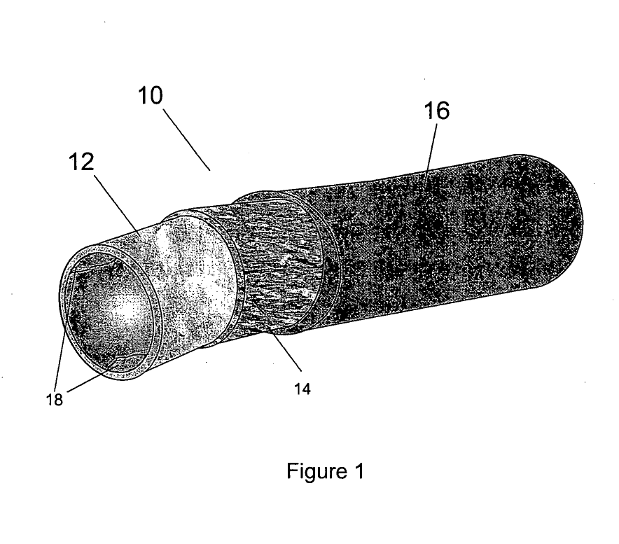

For example, FIG. 1 illustrates a vascular pathway exhibiting narrowing or renarrowing such that a trained physician could identify it as a target site in accordance with the teachings of the present invention. In FIG. 1, vascular pathway 10 is shown comprising three tissue layers consisting of intimal layer 12, medial layer 14 and adventitial layer 16. Vascular pathway 10 exhibits vascular narrowing as evidenced by unwanted cell growth and plaque buildup 18 on the interior surface of intimal layer 12.

Utilizing the teachings of the present invention, trained physicians can identify targets sites in narrowed vascular pathways such as in FIG. 1 diagnostic procedures including, without limitation, X-ray analysis, ultrasound imaging, catheter endoscopic inspection, fluoroscope observation of injected dyes, and digital imaging techniques. One such exemplary digital imaging technique is known as “transesophageal echocardiogram”. Transesophageal echocardiogram uses sound waves to produce real time images of the patient's heart enabling the physician to evaluate its structure and function. This visualization technique provides detailed information on size, shape and movement of the heart muscle in addition to providing the ability to visualize the condition of the patient's vascular pathways, aorta, and coronary arteries. The coronary arteries feed the heart muscle itself. The aorta is the main blood vessel supplying oxygenated blood to the rest of the patient's body.

Utilizing transesophageal echocardiogram to practice the first step of the present invention involves placing a tube-like device called a transducer into the mouth of the patient and guiding it down the patient's esophagus to a position adjacent to the patient's heart. High frequency sound waves, known as “ultrasound”, are delivered through the transducer and pass through the patient's body where they bounce off the patient's heart and echo back to the transducer much like sonar uses sound waves to visualize objects underwater. The sound waves received by the transducer are processed with electronics to create a moving, real time image which is projected onto a monitor which the physician observes to directly visualize the patient's vasculature. The visualized images are then used by the physician to diagnose the patient's coronary arteries for evidence exhibiting vascular narrowing, such as plaque buildup at one or more target sites, or to diagnose the potential or risk of vascular narrowing at one or more target sites, such as tearing, scarring or other injury to the vascular pathway being observed. When such evidence of narrowing or the potential for narrowing is found by the physician and determined to be a target site, then identified target site or sites is then treated with at least one of the compositions of the present invention.

This second step of the present invention involves delivering at least one cell growth inhibiting ubiquitin activator to the one or more identified target sites of the patient. This delivering step can be accomplished through a variety of techniques utilizing the exemplary delivering apparatus, or their equivalents, within the scope and teachings of the present invention. These exemplary delivering apparatus include, without limitation, catheters, stents, microsyringes, endoscopic transport devices syringes, IV's, and drug eluting implants. This is accomplished by the physician positioning such delivery apparatus adjacent to one or more of the identified target sites utilizing placement techniques that are appropriate for the specific delivering apparatus being used within the scope and teachings of the present invention. For example, those skilled in the art will appreciate that, without limitation, diagnostic imaging and target site identification procedures, such as transesophageal echocardiogram techniques, can be used to monitor the placement or positioning of the delivering apparatus.

An example of the preventative or prophylactic percutaneous delivery of one or more compositions of the present invention to an identified target site is illustrated by delivering at least one such composition through the patient's skin into an identified target site in the leg of a patient using a syringe.

Alternatively, in coronary by-pass grafting procedures, venous conduits of the patient are used to by-pass coronary arterial blockages requiring a second surgery performed prior to by-pass grafting to remove a healthy vein from the patient's leg to use as a graft. Utilizing the teachings of the present invention, a trained physician can use a syringe to inject one or more compositions of the present invention through the patient's skin and into the vascular pathway tissues as a preventative measure in order to inhibit vascular narrowing at the identified sites created where the graft vein was removed from the patient's leg.

Additionally, minimally invasive endoscopic or catheter-based microsyringe techniques can be used for delivering at least one cell growth inhibiting ubiquitin activator of the present invention directly into the tissue of the target site or sites within the patient's vasculature from within the vascular pathway itself. Utilizing the teachings of the present invention to compound the medicament compositions of the present invention for injection with appropriate liquid or viscous carriers it is possible to inject the medicament compositions into specific tissues forming the vascular pathway, such as the patient's vascular adventitial layer, in order to inhibit unwanted cell growth within these tissues at the identified target site or sites. The vascular adventitial layer or “adventitia” one of the tissue layers that make up the walls of vascular pathways such as veins or arteries. The adventitia includes a network of small vessels called the “vasa vasorum” that supplies the fluids, nutrients, and oxygenated blood that are critical for the health and function of the vascular pathway itself. As an added advantage of the present invention, delivering the medicament compositions of the present invention with a microsyringe makes it possible to use much smaller dosages of the cell growth inhibiting ubiquitin activators without sacrificing the effectiveness of the present invention. Those skilled in the art also will appreciate that reducing the quantity of pharmaceutical compounds delivered to a patient provides the added benefits of reducing medical costs as well as reducing potential side effects.

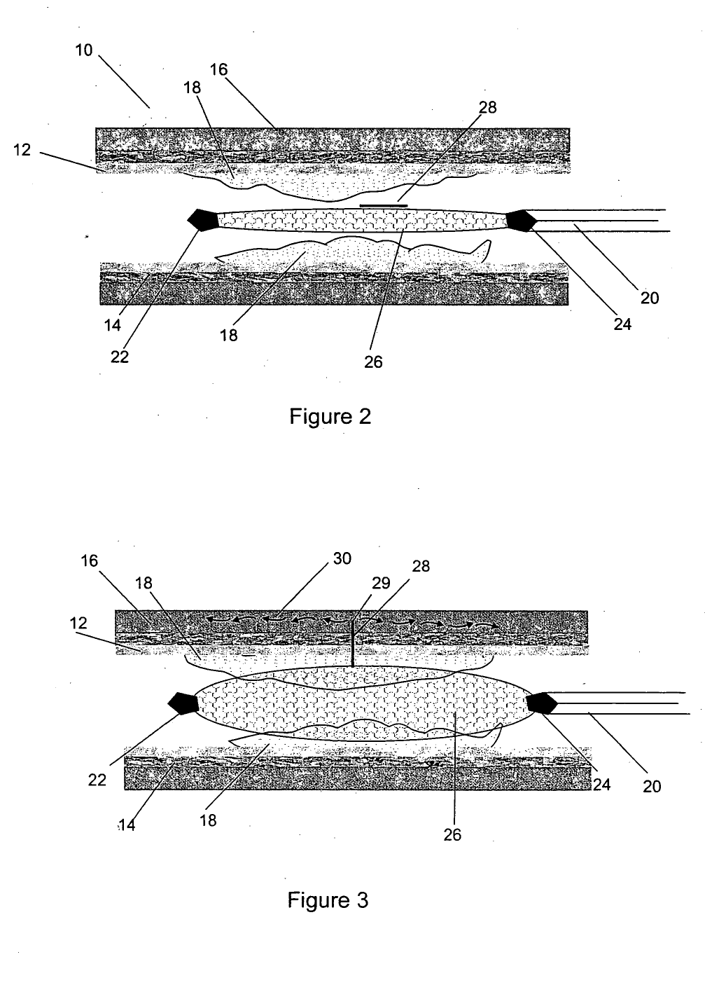

For example, referring to FIG. 2, a partial cross-sectional view of vascular pathway 10 of FIG. 1, vascular pathway 10 exhibits vascular narrowing evidenced by unwanted cell growth and plaque buildup 18 on intimal layer 12. Utilizing the teachings of the present invention, a physician has identified plaque buildup 18 as a target site, and, in accordance with step two of the present invention, a delivery apparatus identified as catheter 20 having actuating balloon 26, with distal end 22 and proximal end 24, has been positioned at the target sites defined by plaque build-up 18. Microsyringe 28 is carried on the surface of actuating balloon 26. As illustrated in FIG. 2, actuating balloon 26 is positioned so that the identified target site, plaque build-up 18, is situated between distal end 22 and proximal end 24 of actuating balloon 26. Alternatively, it is within the scope of the present invention to position actuating balloon 26 such that target site 18 is adjacent to either distal end 22 or proximal end 24 of actuating balloon 26 depending on the judgment of the treating physician.

Referring now to FIG. 3, actuating balloon 26 is shown inflated to both restore the patency or openness of vascular pathway 10 and to at least one delivery of the compositions of the present invention to a specific tissue of the target site 18, in this example the adventitial layer 16 in order to inhibit further vascular narrowing in accordance with the teachings of the present invention. Inflating actuating balloon 26 to an expanded state, causes microsyringe 28 to penetrate through intimal layer 12 and medial layer 14 such that microsyringe tip 29 penetrates adventitial layer 16. Then by pumping a medicament of the present invention through catheter 20 into actuating balloon 26 and out through microsyringe 28 into adventitial layer 16 the physician is able to deliver the exemplary medicament of the present invention into the specific tissue at target site 18 as illustrated by drug flow arrows 30 to inhibit vascular narrowing.

It should be noted that for illustrative purposes, adventitial layer 16 is illustrated as a smooth, flat surface. However, it will be appreciated by those skilled in the art that the adventitial layer may have an irregular shape due to the complex nature of tissues, nerves and blood vessels within the adventitial layer that provide critical support to the vascular pathway itself. Tearing or scarring also may alter the structural or surface appearance of the adventitial layer.

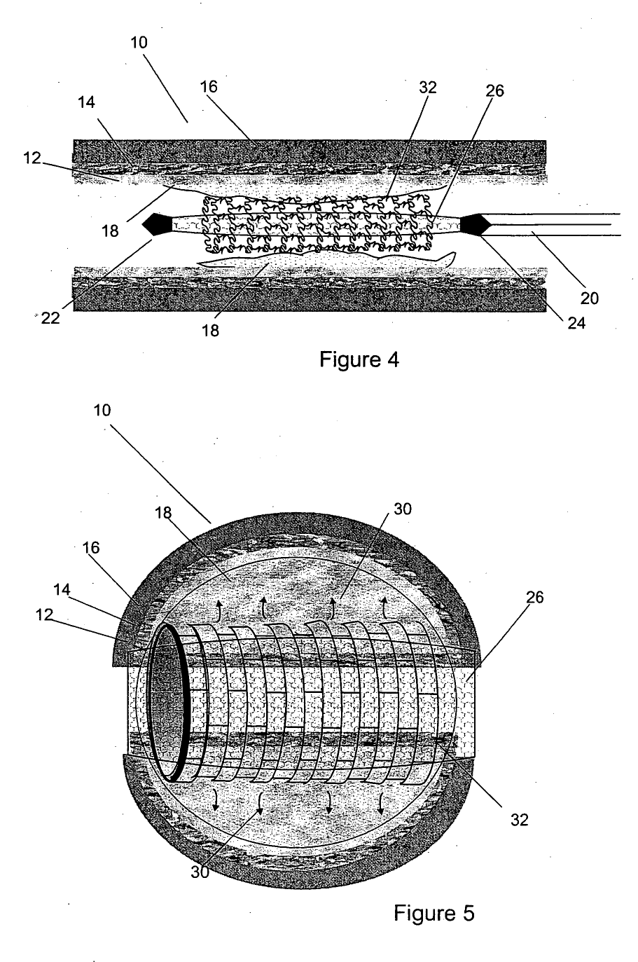

Alternative examples of the methods of the present invention utilize ubiquitin activator delivering apparatus other than exemplary catheter 20 are illustrated in FIGS. 4 and 5. FIG. 4 is a partial cross-sectional view of vascular pathway 10 of FIG. 1 with actuating balloon 26 positioned by catheter 20 at an identified target vascular site defined by plaque build-up 18. In this alternative embodiment of the present invention the ubiquitin acutator delivering apparatus “drug eluting stent” 32 carried on the surface of actuating balloon 26. Drug eluting stent 32 is an example of a delivering apparatus of the present invention having at least one cell growth inhibiting ubiquitin actuator compounded as a medicament with a time release carrier that is coated onto the surface of a stent. Drug eluting stent 32 can also be fabricated having grooves or wells in its surface that function as receptacles or reservoirs for the medicament compositions of the present invention that are delivered over time after drug eluting stent 32 has been expanded into position at or near the identified target site as illustrated in FIG. 5.

FIG. 5 illustrates the delivery of exemplary medicament compositions of the present invention utilizing drug eluting stent 32 is positioned at the identified target site defined by plaque build-up 18 on intimal layer 12. As illustrated in FIG. 5, actuating balloon 26 is inflated to restore the patency of vascular pathway 10 or to position drug eluting stent 32 in a previously opened vascular pathway. Drug eluting stent 32 is expanded to assist in maintaining the openness of vascular pathway 10. The delivering step of the present invention is accomplished when the suitably compounded medicament compositions are released over time from the surfaces of expanded drug eluting stent 32 as illustrated by drug flow arrows 30.

Tissue surface delivering apparatus, such as drug eluting stent 32, utilize medicament compositions of the present invention compounded for release from a drug eluting coating, attachment, grooves or wells associated with such delivering apparatus. In accordance with the teachings of the present invention, these medicament compositions can be compounded in appropriate dosages with dissolving polymers, terpolymers, hydrogels, salts, or other drug eluting compounds that will form stable, time release coatings. Such delivering apparatus can be coated by either spraying the compounded medicament onto the delivering apparatus or by immersing the delivering apparatus into the appropriately compounded medicaments or by other techniques as know in the art or developed in the future.

Application by immersion or spraying may require compounding the medicament to vary the viscosity and surface tension of the compounded medicaments to achieve desired properties. However, spraying in a fine spray such as that available from an airbrush provides both a uniform coating and control over the amount of coating being applied. Further multiple application steps can provide improved coating uniformity and control over the amount of cell growth inhibiting ubiquitin activator medicament compositions being applied. Moreover, in an alternative exemplary embodiment of the present invention, the cell growth inhibiting ubiquitin activator medicaments can be applied in a base coat on a drug eluting stent, followed by a topcoat having differing compositions applied over the base coat to vary or control delivering the compositions to of the present invention identified target sites.

It should be emphasized that the alternate embodiments of the present invention can include injection techniques, such as the use of microsyringe delivering apparatus, and tissue surface delivering techniques, such as the use of drug eluting stent apparatus, as separate, combined or sequential delivering steps at the identified target sites. Similarly, the methods of the present invention can be practiced in conjunction with the restoration of vessel patency utilizing balloon angioplasty and stent positioning, or as separate steps that are completely independent of such vessel reopening procedures.

The cell growth inhibiting ubiquitin activator medicament compositions of the present invention are selected from the group consisting of protein kinase inhibitors, tyrosine-specific kinase inhibitors, deubiquitination enzyme inhibitors, antibiotics, antifungal agents, anti-tumor agents, and derivative products thereof. Thus, it should be emphasized that degradation products and derivative products of the compositions of the present invention are within the scope of the present invention.

For example, hypothemycin is a cell growth inhibiting ubiquitin activator useful in practicing the present invention. Hypothemycin is known in the art as an antibiotic and a tyrosine kinase inhibitor, as well as an antifungal agent. It is a metabolite of Hypomyces trichothecoides that is active against Tetrahymena furgasoni and Ustilago maydis and also exhibits anti-tumor activity on laboratory tumor cell lines. In accordance with the teachings of the present invention, hypothemycin is utilized as a cell growth inhibiting ubiquitin activator that is delivered to one or more target sites that have been identified as being at risk of narrowing or exhibiting vascular narrowing.

Once released at an identified target site or sites using delivering apparatus such as those disclosed as being within the scope of the present invention, or their equivalents, hypothemycin inhibit functions to cell growth by reacting with at least one biologic factor involved with biological cell growth mechanisms. One such biologic factor is “ubiquitin” and is commonly found in virtually all mammalian cells. Ubiquitin is involved in regulating the degradation of proteins over time, which is a metabolic process known as “protein turnover”. Ubiquitin functions to inhibit unwanted cell growth within the teachings of the present invention by closely regulating the degradation of cell growth-promoting proteins such as cyclin D1 proteins. This elimination or degradation of cyclin D1 proteins results in the inhibition, restraining, or even in the prevention of unwanted cell growth that is normally promoted by cyclin D1 proteins.

Those skilled in the art will appreciate that at target vascular sites exhibiting narrowing or the potential risk of narrowing there is a need to specifically inhibit unwanted cell growth without negatively impacting tissues at other parts of the patient's body. This can be accomplished through the teachings of the present invention by inhibiting or restraining the ability of cell growth factors to induce unwanted cell growth in specific tissues, such as the adventitial layer, at such identified target vascular sites.

A further understanding of the present invention will be provided to those skilled in the art from the following exemplary discussion illustrating the relationship between ubiquitin, protein degradation enzymes, and cell growth-promoting proteins such as cyclin D1 proteins, as related to the inhibition of cell growth factors provided by the present invention. Particularly, the roles ubiquitin demonstrates relative to the elimination or degradation of cell growth promoting cyclin D1 proteins by enzyme activities.

Ubiquitin itself does not degrade cyclin D1 proteins, but rather functions as a reaction tag that marks cyclin D1 proteins for degradation or elimination by an enzyme called “proteasome”. This is in an ATP dependent function where activating enzymes hydrolyze ATP as part of the protein tagging process. The multiple tagging of cyclin D1 proteins by ubiquitin is necessary for the recognition of target proteins by proteasome enzymes. This multiple tagging by ubiquitin, known as “polyubiquitination”, increases the molecular weight of the ubiquitin-cyclin D1 protein conjugated complexes when compared to unconjugated cyclin D1 proteins.

Individual ubiquitin molecules or ubiquitin chains are covalently conjugated to cyclin D1 proteins through a bond between the glycine at the C-terminal end of ubiquitin and the side chains of lysine on cyclin D1 proteins. The conjugation process is dependent on the hydrolysis of ATP. Once conjugated, cyclin D1 proteins can now be recognized and bound to the ubiquitin receptors of proteasome enzymes.

The proteasome enzymes are made up of many different proteases and have key roles in the metabolizing and degradation of proteins, such as growth promoting cyclin D1 proteins. It is the 26S form of proteasome that recognizes cell growth promoting cyclin D1 proteins that have been polyubiquitinated. The 26S form of proteasome consists of two 19S regulators on its 20S catalytic core. More specifically, the 19S regulator core has ubiquitin chain receptors that recognize polybubiquitinated or multiple ubiquitin tagged cyclin D1 proteins. The ubiquitin tagging and proteasome degrading process is called the “ubiquitin-proteasome pathway”.

The ubiquitin-proteasome pathway utilizes three enzymes starting with E1 enzymes known as “ubiquitin-activating enzymes” that modify ubiquitin so it is in a reactive state and able to conjugate to cyclin D1 proteins. This increases the likelihood that the C-terminal glycine on ubiquitin will react with the lysine side-chains on cyclin D1 proteins. Then, E2 enzymes, known as “ubiquitin-conjugating enzymes”, catalyze the attachment of ubiquitin to the cyclin D1 proteins. At this point, E3 enzymes known as “ubiquitin-ligases” function in concert with E2 enzymes to provide targeting mechanisms for 26S proteasome enzymes that degradate or eliminate cyclin D1 proteins. The degredation or elimination of cyclin D1 proteins leads to the inhibition of cell growth.

More specifically, the ubiqutin-proteasome pathway utilization of the three E1 enzymes in the degradation or elimination of cyclin D1 proteins in order to inhibit cell growth related to vascular narrowing or renarrowing first involves the E1 enzyme hydrolysis of ATP. This forms complexes with the resulting ubiquitin adenylate similar to the amino acyl adenylate formation in protein synthesis. Transfer of ubiquitin follows this reaction to the active site cysteine of E1 to form a thiol ester between the C-terminus of ubiquitin and the thiol group of E1. This transfer occurs in concert with adenylation of an additional ubiquitin.

Thiol esters are readily cleaved by reducing agents such as mercaptoethanol and dithiothreitol and also by hydroxylamine. These reactions are illustrated by the following equations:

$$\begin{matrix}E1 + ATP + ubiquitin ⟶ {E1}^{. Ub \text{-} AMP} + PPi \\ {E1}^{. Ub \text{-} AMP} + ubiquitin ⟶ {E1}_{\text{-} s \text{-} co \text{-} Ub}^{. AMP \text{-} Ub}\end{matrix}$$

Ubiquitin is not transferred from E1 to a target cyclin D1 protein, but rather is transferred to one of a family of ubiquitin-conjugation enzymes or ligases (E2). These enzymes are proximal donors of ubiquitin to target cyclin D1 proteins. E2 enzymes also have an active site cysteine, and ubiquitin is transferred from E1 to the E2 cysteine to form a thiol ester as illustrated by the following equation:

$${E1}_{\text{-} s \text{-} co \text{-} Ub}^{. AMP \text{-} Ub} + E2 \text{-} SH ⟶ {E2}_{\text{-} s \text{-} co \text{-} Ub} + {E1}^{. AMP \text{-} Ub}$$

Ubiquitin is then transferred to the acceptor lysine of the target cyclin D1 protein to form the isopeptide bond as illustrated by the following equation:

$${E2}_{\text{-} s \text{-} co \text{-} Ub} + cyclin D1 \text{-} {NH}_{2} ⟶ E2 \text{-} SH + cyclin D1 \text{-} NH \text{-} CO \text{-} Ub$$

Multi-ubiquitin chains can be built up on a single lysine of target cyclin D1 protein, by isopeptide bond formation between the carboxyl groups of gly₇₆ of one ubiquitin with the amino group of the side chain of lys₄₈ of the preceding ubiquitin. However, multi-ubiquitin chains containing isopeptide bonds gly₇₆-lys₁₁ are generated by an E2 enzymes in keratinocytes, and these chains are also able to target cyclin D1 proteins for degradation by 26S proteasome.

Multi-ubiquitin chains exhibit certain structural characteristics that are recognized by the proteasome 19S receptor complex. Once the polyubiquitinated cyclin D1 protein complexes are recognized, the next step of the ubiquitin-proteasome pathway is the binding of these complexes to the ubiquitin receptor complex in the 19S regulator. Unraveling of the polyubiquitinated cyclin D1 protein complexes, which is ATP driven, then begins and the complexes are threaded through the 20S core of the 26S proteasome. At this point, the polyubiquitin chains are cleaved and the cyclin D1 proteins are disassociated into smaller products such as peptides or single amino acids. The cleaved ubiquitin molecules can be used or recycled again in the tagging of other cyclin D1 proteins.

Once the cyclin D1 proteins are disassociated by the 26S proteasome, the cell growth promoting or inducing activity of the cyclin D1 proteins are eliminated or degraded, thus inhibiting cell growth. The ATP driven ubiquitin-proteasome pathway process is an important factor in the inhibition of unwanted cell growth related to vascular pathway narrowing or renarrowing promoted by cyclin D1 proteins as disclosed and claimed by the present invention.

The regulation of cyclin D1 proteins controls the progression of unwanted cell growth. Cyclin D1 proteins play a central role in G1 progression in unwanted mammalian cell growth and its expression is stimulated by mitogenic signals. Following these signals, expressed cyclin D1 proteins in G1 phase assembles with cyclin dependent kinases to form active kinase complexes. The amount of complexes produced is titrated by cyclin-dependent inhibitors. Cyclin D1 proteins are induced as the cells proliferate and are dependent on cell growth-promoting enzymes such as tyrosine kinases. These kinases promote the transfer of the terminal phosphate of ATP for use in cell growth processes. Thus, the cell growth inhibiting ubiquitin activators of the present invention include inhibitors of tyrosine kinases as they also inhibit unwanted cell growth.

The antibiotic hypothemycin also functions as an inhibitor of tyrosine kinase by regulating the enzyme's reactions with ATP. The inhibition of tyrosine kinase in accordance with the teachings of the present invention results in the restraining of growth promoting cyclin D1 protein inducement that in turn results in the inhibition of unwanted cell growth. Additionally, the inhibition of tyrosine kinase activities by hypothemycin makes more ATP available for the ubiquitin-proteasome pathway elimination or degradation of cyclin D1 proteins. Thus, in accordance with the teachings of the present invention, hypothemycin contributes to the restraining, degrading or elimination of cyclin D1 proteins making it a particularly illustrative example of the cell growth inhibiting ubiquitin activator medicament compositions of the present invention.

The following examples, without limitation, illustrate the production and manufacture of exemplary delivering apparatus that are within the scope the present invention. It is understood that there are numerous other apparatus embodiments that are within the methods of the present invention that will be apparent to those of ordinary skill in the art after having read and understood this specification. Moreover, it is to be understood that hypothemycin is one example of a cell growth inhibiting ubiquitin activator that is used according to the teachings and scope of the present invention and that other compounds having equivalent functions are within the scope of the present invention.

### EXAMPLE 1

### COATING A CLEAN, DRIED STENT USING A CELL GROWTH INHIBITING UBIQUITIN ACTIVATOR/POLYMER SYSTEM

250 μg of cell growth inhibiting ubiquitin activator or “activator” is carefully weighed and added to a small neck glass bottle containing 27.56 ml of tetrahydofuran (THF). The activator-THF suspension is thoroughly mixed until a clear solution is achieved.

251.6 mg of polycaprolactone (PCL) is added to the activator-THF solution and mixed until the PCL dissolves forming a cell growth inhibiting ubiquitin activator/polymer medicament solution.

Stainless steel stents are placed a glass beaker and covered with reagent grade or better hexane. The beaker containing the hexane-immersed stents is placed into an ultrasonic water bath and treated for 15 minutes at a frequency of between approximately 25 to 50 KHz. Next the stents are removed from the hexane and the hexane discarded. The stents are then immersed in reagent grade or better 2-propanol. The vessel containing the stents and the 2-propanol is treated in an ultrasonic water bath. Following cleaning of the stents with organic solvents, they are thoroughly washed with distilled water and thereafter immersed in 1.0 N sodium hydroxide solution and treated in an ultrasonic water bath. Finally, the stents are removed from the sodium hydroxide, thoroughly rinsed in distilled water and dried in a vacuum oven over night at 40° C.

After cooling the dried stents to room temperature in a desiccated environment they are weighed and their weights were recorded.

The cleaned, dried stents are coated by either spraying or by dipped into the cell growth inhibiting ubiquitin activator/polymer medicament solution. The stents are coated to achieve a final coating weight of between approximately 10 μg to 1 mg. Finally, the coated stents are dried in a vacuum oven at 50° C. over night. The dried, coated stents are weighed and the weights recorded.

The concentration of cell growth inhibiting ubiquitin activator loaded onto the stents is determined based on the final coating weight. Final coating weight is calculated by subtracting the stent's pre-coating weight from the weight of the dried, coated stent.

### EXAMPLE 2

### ALTERNATIVE DRUG-ELUTING VASCULAR STENT INCORPORATING HYPOTHEMYCIN

Hypothemycin is compounded with a biocompatible carrier which is coated onto a drug eluting stent or catheter that is to be used in the treatment of restenosis. First, hypothemycin is dissolved or suspended in any carrier compound that provides a stable composition that does not react adversely with the delivering apparatus to be coated and does not inactivate the hypothemycin as in Example 1.

This medicament compositiion is coated onto a stent coating using any coating technique known to those skilled in the art of medical device manufacturing. Suitable non-limiting examples of coating techniques include impregnation, spraying, brushing, dipping and rolling.

After the hypothemycin compounded medicament compositions are applied to coat the stent, the stent is dried to form a delivering apparatus. Drying techniques include, but are not limited to, heated forced air, cooled forced air, vacuum drying, and static evaporation.

A topcoat can be applied, if desired, over the cell growth inhibiting ubiquitin activator containing base coat to control the delivering or releasing rate of the cell growth inhibiting ubiquitin activator onto the tissue at the target vascular site.

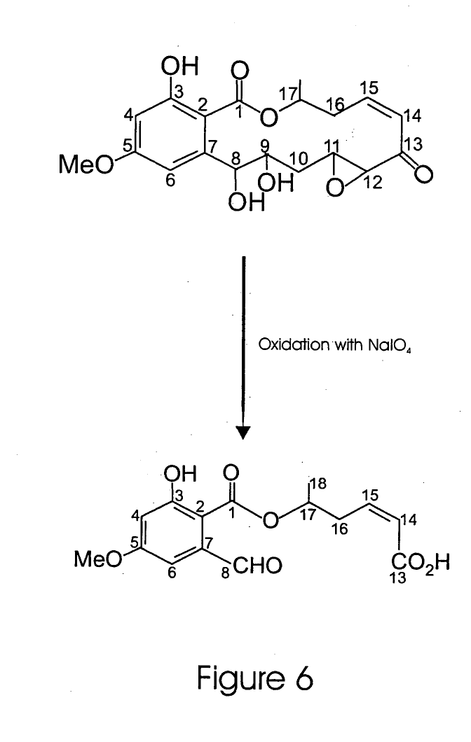

A further understanding of hypothemycin, an exemplary growth inhibiting ubiquitin activator according to the teachings of the present invention, is provided by FIG. 6, an illustration of the chemical structure of hypothemycin and a related derivative product, taken in conjunction with the following discussion of additional chemical and structural aspects of hypothemycin.

### EXAMPLE 3

### EXEMPLARY STRUCTURES OF (+) HYPOTHEMYCIN AND RELATED DEGRADATION PRODUCT

Hypothemycin is isolated from a strain of Hypomyces trichothecoides. The fungus is grown in a dextrose-yeast medium in still culture in the dark at 25° C. producing an antibiotic metabolite. This antibiotic is hypothemycin, C₁₉H₂₂O₈ (elemental analysis); MW 378 (ms) had mp. 173-4°, [α] 365=+109° (0.136% MeOH), CD curve (MeOH) [θ] 335 (+3.351) [θ] 305 (−8,987) [θ] 262 (−40,200) [θ] 234 (+29,600), and [θ] 212 nm (+77,600), λ^{MeOH}ₘₐₓ 220 (38,000), 267 (14,000) and 307 nm (7000), Vₘₐₓ (KBr)˜3350 (b) 1695, 1653, 1620, 1593 and 1250 cm⁻¹. UV and IR spectra suggested a resorcylic acid macrolide structure. In agreement with this, its ¹³C NMR spectrum showed signals at δ 21.0 (C—Me), 34.6 nd 36.9 (two methylenes), six SP₃ carbons carrying oxygen at 55.5 (OMe) 57.9 (C₁₁), 62.6 (Cg) 0.7 (C₁₂), 73.2 (C₈), and 81.0 (C₁₇), and sp₂ carbons at 101.1 (C₄), 103.6 (C₆), 104.0 (C₂), 126.4 (C₁₅), 142.3 (C₁₄), 145.3 (C₇), 165.2 (C₅), 166.2 (C₃), 171.2 (COO) and 199.9 (C₁₃). ¹H NMR spectrum showed 3 protons exchangeable with D₂0. Thus, three hydroxylis, one OMe, one lactone moiety and one carbonyl, account for seven of the eight oxygens. The eighth oxygen has to be in an oxiran ring to explain the ¹³C NMR, as well as the molecular formula.

On oxidation with NalO₄ in aqueous methanol, hypothemycin gave an aldehyde acid derivative or degredation product, mw 308 (ms). ¹H NMR spectrum (CDCL₃) of this acid showed signals at δ 1.41 (3H, J=6.5) for the C—Me, 3.1 (2H dd J=6.5,8) for the C₁₆ protons, 3.83 (3H, s) for the OMe, 5.45 (1H, q t J=6.5,6.5) for the C₁₇ proton, 5.95 (1H, dd J=11) for the C₁₄ proton, 6.4 (1H, dd J=8,11) for the C₁₅ proton and an AB quartet at 6.6 and 6.8 (J=2) for the aromatic protons. Formation of this degradation product defined all but four of the carbons in the macrolide ring. Detailed analysis of ¹H NMR using sequential decoupling defined the structure of hypothemycin as illustrated in FIG. 6. NMR signal of protons on carbons 8 through 12 were: 8H, δ 4.58, dd (J_{8,OH}^{=4.5}; J_{8,9}^{=2.0}) 9H, δ 3.93 m (J_{9,OH}⁼⁹; J_{9,8}^{=2.0}; J_{9,10}⁼⁴; J_{9,10}^{=8.7}) 10H, δ 1.12 m (J_{10,10′}⁼¹⁵; J_{10,9}⁼⁴; J_{10,11}^{=8.7}), 10′H, δ 2.05 J_{10′10}⁼¹⁵; J_{10′11}⁼²; J_{10′9}^{=8.7}) 11H, δ 2.89 m (J_{11,12}⁼²; J_{11,10′}⁼²) 12H 4.41 d (J_{12,11}⁼²). The phenolic proton signal at 12.1 showed that it was chelated to the lactone carbonyl. A coupling constant of 2 Hz between the aromatic protons showed them to meta to each other. Therefore, the OH and OMe are on carbons 3 and 5, respectively

The coupling constant of 11 Hz between the olefinic protons in hypothemycin showed them to be cis since the trans coupling constant in comparable 7-dehydro zearalenone is 16 Hz. The degradation product illustrated in FIG. 6 also showed J=11 between the ofefinic protons confirming this assignment. The coupling constant of 2 Hz between the oxiran ring protons showed them to be trans; in comparable radicicol the trans coupling constant is reported as 3.0 and 2.8 Hz. In oxiran rings the cis coupling constant is always much larger than the trans coupling constant. The vicinal hydroxyls in the lactone ring are threo, since the coupling constant of the protons on those carbons is 2 Hz.

The mass spectrum is in complete agreement with this hypothemycin structure. The major fragments are at m/e 180 (80%) and 179 (100%) formed by cleavage of bonds at C₈ to C₉ and C₁.

Using the preceding detailed description and examples, it is possible for one of ordinary skill in the art of polymer chemistry to design medicament compositions and coatings having a wide range of dosages, administration, and solubility rates that are within the scope of the present invention. Without limitation, drug delivering rates and concentrations can also be controlled using non-polymer containing coatings and techniques known to persons skilled in the art of medicinal chemistry and medical device manufacturing protocols.

It should also be appreciated by those skilled in the art that by defining and developing various solubility rates of the cell growth inhibiting ubiquitin activator compositions of the present invention, those skilled in the art of polymer chemistry will be able to design universal delivering apparatus that are within the scope of the present invention.

In addition, the cell growth inhibiting ubiquitin activator medicament compositions and methods of the present invention, without limitation, can be used in research, medical product manufacturing, or administered as prophylactic or therapeutic treatments or therapies in mammals and humans in accordance with the appropriate research, clinical trial, manufacturing or treatment protocols or procedures approved by the appropriate governing institutions having authority to recommend, approve, evaluate or regulate such protocols or procedures.

Further, without limitation, the cell growth inhibiting ubiquitin activator medicament compositions of the present invention are delivered to identified target vascular sites using a broad range of dosages. Such a broad range of dosages includes, without limitation, the highest non-toxic or minimally toxic concentrations determined or established by trained medical professionals or researchers for compositions of the present invention tested, used or applied in mammals and humans.

Unless otherwise indicated, all numbers expressing quantities of ingredients, properties such as molecular weight, reaction conditions, and so forth in the specification and embodiments are to be understood as being modified in all instances by the term “about.” Accordingly, unless indicated to the contrary, the numerical parameters set forth in the specification are approximations that may vary depending upon the desired properties sought to be obtained by the present invention. At the very least, and not as an attempt to limit the application of the doctrine of equivalents to the scope of the embodiments, each numerical parameter should at least be construed in light of the number of reported significant digits and by applying ordinary rounding techniques. Notwithstanding, numerical ranges and parameters setting forth the broad scope of the invention are approximations, the numerical values set forth are reported as precisely as possible. Numerical values, however, inherently contain certain errors necessarily resulting from the standard deviation found in their respective testing measurements.

The terms “a” and “an” and “the” and similar referents are used in the context of describing the invention and are to be construed to cover both the singular and the plural, unless otherwise indicated herein or clearly contradicted by context. Recitation of ranges of values herein are merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illustrate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended embodiments.

Preferred embodiments of this invention are described herein, including the best mode known to the inventors for carrying out the invention. Of course, variations on those preferred embodiments will become apparent to those of ordinary skill in the art upon reading the foregoing description. Accordingly, this invention includes all modifications and equivalents of the subject matter recited in the embodiments appended hereto as permitted by applicable law. Moreover, any combination of the above-described elements in all possible variations thereof is encompassed by the invention unless otherwise indicated herein or otherwise clearly contradicted by context.

In closing, it is to be understood that the embodiments disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A method for inhibiting vascular narrowing in a mammal, said method comprising the steps of:
identifying at least one target vascular site in said mammal; and
delivering at least one cell growth inhibiting ubiquitin activator to said target vascular site.

2. The method of claim 1 wherein said target vascular site is at risk of narrowing.

3. The method of claim 1 wherein said delivering step further comprises delivering said at least one cell growth inhibiting ubiquitin activator directly to the tissue at said target vascular site.

4. The method of claim 3 wherein said tissue of said target vascular site is the vascular adventitial layer.

5. The method of claim 4 wherein said delivering step comprises positioning a cell growth inhibiting ubiquitin activator delivering apparatus at said target vascular site.

6. The method of claim 5 wherein said growth inhibiting ubiquitin activator delivering apparatus is a catheter.

7. The method of claim 5 wherein said cell growth inhibiting ubiquitin activator delivering apparatus is a stent.

8. The method of claim 5 wherein said cell growth inhibiting ubiquitin activator delivering apparatus is a microsyringe.

9. The method of claim 8 wherein said delivering step comprises at least one injection directly into said tissue at said target vascular site.

10. The method of claim 1 wherein said at least one cell growth inhibiting ubiquitin activator is selected from the group consisting of protein kinase inhibitors; tyrosine-specific kinase inhibitors; deubiquitination enzyme inhibitors; antibiotics; antifungal agents; anti-tumor agents and derivative products thereof.

11. The method of claim 1 wherein said at least one cell growth inhibiting ubiquitin activator is hypothemycin.

12. A medicament for inhibiting vascular narrowing in a mammal, said medicament comprising an effective amount of at least one cell growth inhibiting ubiquitin activator and a carrier.

13. The medicament of claim 12 wherein said carrier is compounded for injection.

14. The medicament of claim 12 wherein said carrier is compounded for release from a delivering apparatus positioned at said target vascular site.

15. The medicament of claim 12 wherein said at least one cell growth inhibiting ubiquitin activator is selected from the group consisting of protein kinase inhibitors; tyrosine-specific kinase inhibitors; deubiquitination enzyme inhibitors; antibiotics; antifungal agents; anti-tumor agents, and derivative products thereof.

16. The medicament of claim 15 wherein said at least one cell growth inhibiting ubiquitin activator is hypothemycin.

17. A vascular stent comprising a controlled release coating that provides an anti-proliferative amount of at least one cell growth inhibiting ubiquitin to a specific site in the vasculature of a mammal.

18. The vascular stent according to claim 17 wherein said at least one cell growth inhibiting ubiquitin activator is hypothemycin.

19. The vascular stent according to claim 17 further comprising a controlled release polymer coating comprising at least one terpolymer and at least one co-polymer.

20. A vascular stent comprising a controlled release polymer coating comprising a terpolymer and at least one copolymer wherein said controlled release polymer controls the release of hypothemycin.
